(19) United States
(12) Patent Application Publication
(10) Pub. No.: US 2011/0110817 A1
(43) Pub. Date: May 12, 2011

(54) RARE EARTH REMOVAL OF COLORANTS

(75) Inventors: Joseph A. Lupo, Henderson, NV (US); Joseph R. Pascoe, Las Vegas, NV (US)

(73) Assignee: MOLYCORP MINERALS, LLC, Greenwood Village, CO (US)

(21) Appl. No.: 12/942,847

(22) Filed: Nov. 9, 2010

Related U.S. Application Data
(60) Provisional application No. 61/259,314, filed on Nov. 9, 2009.
(60) Provisional application No. 61/386,347, filed on Sep. 24, 2010.
(60) Provisional application No. 61/386,365, filed on Sep. 24, 2010.
(60) Provisional application No. 61/386,394, filed on Sep. 24, 2010.

(51) Int. Cl. A61L 2/12; C01F 17/00; A61L 2/10; A61L 2/08; A61L 2/16; C09K 3/00; C09B 62/00; C09B 67/44; C09C 1/62; C08K 3/08

(52) U.S. Cl. 422/21; 423/263; 422/24; 422/22; 422/28; 422/292; 252/182.33; 8/543; 8/618; 106/403; 524/440

(57) ABSTRACT

The present disclosure is directed to a method and system for contacting a colorant-contaminated aqueous solution with a rare earth-containing composition to form a treated solution substantially depleted of the colorant.

### CROSS REFERENCE TO RELATED APPLICATION

The present application claims the benefits of U.S. Provisional Application Ser. No. 61/259,314, filed Nov. 9, 2009, entitled “Removal of Water Soluble Organic Dyes Using Cerium Oxide”; 61/386,347, filed Sep. 24, 2010, entitled “Removal of a Dye From An Aqueous System Using Rare Earths”; 61/386,365, filed Sep. 24, 2010, entitled “Removal of a Colorant From An Aqueous System Using Rare Earths”; and 61/386,394, filed Sep. 24, 2010, entitled “Removal of a Pigment From An Aqueous System Using Rare Earths”, each of which is incorporated herein by this reference in its entirety.

### FIELD

The invention relates generally to removal of colorants and particularly to rare earth removal of colorants from aqueous streams.

### BACKGROUND

Colorants are used in a wide variety of applications. Colorants, for example, are used in food products, textiles, personal care products, paints, and a host of other products. Colorants typically comprise a dye or a pigment.

The scale and growth of the dye industry is inextricably linked to that of the textile industry. World textile production has grown steadily to an estimated 35 million tons in 1990, and the estimated production of dyes in 2009 was around 120 million tons.

With the increased demand for textile products, the textile industry and its wastewaters have been increasing proportionately, making it one of the main sources of water pollution worldwide. The release of colorant-containing effluents into the environment is undesirable, not only because of their color but also because many colorants and their breakdown products are toxic and/or mutagenic to life. Many of the colorants are chemically stable and without adequate treatment they can remain in the environment for an extended period of time. Furthermore, many colorants in particular pigments comprise heavy metals. In addition to environmental pollution, the textile industry contributes to water shortage by consuming large amounts of potable water. In many countries where potable water is scarce, this large water consumption has become intolerable and wastewater recycling has been recommended to decrease the water requirements.

As a consequence of these issues, increasingly stringent government air and water pollution control regulations are restricting colorant use. Various organizations and/or agencies, as for example, the American Organization of Analytical Chemists, the World Health Organization, and the US Environmental Protection Agency have addressed the issue of air and water pollution arising from colorant usage.

However, removal of colorants from aqueous streams can be difficult.

One option to remove colorants from water streams is to add expensive chemicals that destroy or react with the colorant. Chemical oxidation uses a suitable oxidizing agent, such as ozone, hydrogen peroxide, or permanganate to change the chemical composition of a colorant, such as a dye.

Another option is to pass the colorant-containing aqueous stream through an expensive ultrafilter, nanofilter, and/or reverse osmosis membrane system to separate the colorant from the water. Typically, the separation is based on molecular size.

Yet another option is to use physical-chemical colorant removal methods. Coagulant agents, such as ferric salts and aluminum polychloride, form flocks with the colorants, which are then separated by filtration or sedimentation.

Still yet another option is to biologically degrade the colorant with a microbe under aerobic and/or anaerobic conditions. Typically the microbes are bacteria or fungi.

Another option is to remove the colorant by an adsorption process. Colorants, such as dyes have high affinities for adsorbent materials.

Although many techniques have been used to remove colorants, there is no single and economically attractive treatment that can effectively remove and/or decolorize dyes and their decomposition products.

### SUMMARY

These and other needs are addressed by the various embodiments and configurations of the present invention. The invention relates generally to the removal of a selected component of a colorant composition from a contaminated aqueous solution.

In an embodiment, a treatment method is provided that contacts a colorant-containing aqueous solution with a rare earth-containing composition to form a treated solution substantially depleted of the colorant.

In an embodiment, a treatment system is provided that includes:

an inlet for a colorant-containing aqueous solution;

a rare earth-containing composition in a colorant removal zone; and

an outlet for a treated aqueous solution substantially depleted of the colorant.

In an embodiment, the method and/or system produce a contaminated rare earth-containing composition including the rare earth and the colorant sorbed on the rare earth. In other embodiments, the colorant is degraded or decomposed by the rare earth, such as by destruction of a chromophore.

In an embodiment, a regeneration method is provided that includes the steps:

(a) providing a contaminated rare earth-containing composition comprising a rare earth and a colorant sorbed on the rare earth and

(b) sterilizing the contaminated rare earth-containing composition to remove the colorant.

The embodiments can provide a number of advantages depending on the particular configuration. For example, in many applications rare earth-containing compositions can economically and effectively decolorize or remove colorants. Accordingly, the embodiments can overcome the environmental problems associated with colorant discharge and enable wastewater recycling to decrease water requirements of the textile industry. The rare earth-containing composition can treat colorant contaminated aqueous solutions to contamination levels in compliance with the increasingly stringent air and water pollution control regulations by private and governmental organizations, such as the American Organization of Analytical Chemists, the World Health Organization, and the US Environmental Protection Agency.

These and other advantages will be apparent from the disclosure of the invention(s) contained herein.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The terms “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁—Xₙ, Y₁—Yₘ, and Z₁—Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

“Absorption” refers to the penetration of one substance into the inner structure of another, as distinguished from adsorption.

“Adsorption” refers to the adherence of atoms, ions, molecules, polyatomic ions, or other substances of a gas or liquid to the surface of another substance, called the adsorbent. The attractive force for adsorption can be, for example, ionic forces such as covalent, or electrostatic forces, such as van der Waals and/or London's forces.

“Anthraquinone” refers to a substance based on 9,10-anthraquinone (which is essentially colorless) having an electron-donor group, such as amino or hydroxyl introduced into one or more of the four alpha positions (1, 4, 5, and 8).

An “auxochrome” is a chemical substitute that intensifies the color of a chromophore by withdrawing or donating electrons to the chromophore. Common auxochrome substituents include amine (—NH₃), carboxyl (—C(═O)OH), sulfonate (—SO₃H), and hydroxyl (—OH).

A “chromophore” is a group of atoms responsible for the dye color. Examples of chromophores are azo (—N═N—), carbonyl (>C═O), methine (═(C—H)—), nitro (—NO₂), hydrazo (the bivalent group —HNNH—), anthraquinone, alkyne (HC≡), styryl (C₆H₅—CH═C<), methyl (—CH₃), cyanine, thiazine, and quinone.

A “colorant” is any substance that imparts color, such as a pigment or dye.

A “composition” refers to one or more chemical units composed of one or more atoms, such as a molecule, polyatomic ion, chemical compound, coordination complex, coordination compound, and the like. As will be appreciated, a composition can be held together by various types of bonds and/or forces, such as covalent bonds, metallic bonds, coordination bonds, ionic bonds, hydrogen bonds, electrostatic forces (e.g., van der Waal's forces and London's forces), and the like.

The term “contained within water” refers to materials suspended and/or dissolved within the water. The suspended material has a particle size. Suspended materials are substantially insoluble in water and dissolved materials are substantially soluble in water.

“De-toxify” or “de-toxification” includes rendering a chemical contaminant non-toxic to a living organism, such as, for example, human or other animal. The chemical contaminant may be rendered non-toxic by converting the contaminant into a non-toxic form or species.

A “dye” is a colorant, usually transparent, which is soluble in an application medium. Dyes are classified according to chemical structure, usage, or application method. They are composed of groups of atoms responsible for the dye color, called chromophores, and intensity of the dye color, called auxchromes. The chemical structure classification of dyes, for example, uses terms such as azo dyes (e.g., monoazo, disazo, trisazo, polyazo, hydroxyazo, carboxyazo, carbocyclic azo, heterocyclic azo (e.g., indoles, pyrazolones, and pyridones), azophenol, aminoazo, and metalized (e.g., copper (II), chromium (III), and cobalt (III)) azo dyes, and mixtures thereof), anthraquinone (e.g., tetra-substituted, disubstituted, trisubstituted and momosubstitued, anthroaquinone dyes (e.g., quinolines), premetallized anthraquinone dyes (including polycyclic quinones), and mixtures thereof), benzodifuranone dyes, polycyclic aromatic carbonyl dyes, indigoid dyes, polymethine dyes (e.g., azacarobocyanine, diazacarbocyanine, cyanine, hemicyanine, and diazahemicyanine dyes, triazolium, benothiazolium, and mixtures thereof), styryl dyes, (e.g., dicyanovinyl, tricyanovinyl, tetracvanoctylene dyes) diaryl carbonium dyes, triaryl carbonium dyes, and heterocyclic derivates thereof (e.g., triphenylmethane, diphenylmethane, thiazine, triphendioxazine, pyronine (xanthene) derivatives and mixtures thereof), phthalocyanine dyes (including metalized phthalocyanine dyes), quinophthalone dyes, sulfur dyes, (e.g., phenothiazonethianthrone) nitro and nitroso dyes (e.g., nitrodiphenylamines, metal-complex derivatives of o-nitrosophenols, derivatives of naphthols, and mixtures thereof), stilbene dyes, formazan dyes, hydrazone dyes (e.g., isomeric 2-phenylazo-1-naphthols, 1-phenylazo-2-naphthols, azopyrazolones, azopyridones, and azoacetoacetanilides), azine dyes, xanthene dyes, triarylmethane dyes, azine dyes, acridine dyes, oxazine dyes, pyrazole dyes, pyrazalone dyes, pyrazoline dyes, pyrazalone dyes, coumarin dye, naphthalimide dyes, carotenoid dyes (e.g., aldehydic carotenoid, β-carotene, canthaxanthin, and β-Apo-8′-carotenal), flavonol dyes, flavone dyes, chroman dye, aniline black dye, indeterminate structures, basic dye, quinacridone dye, formazan dye, triphendioxazine dye, thiazine dye, ketone amine dyes, caramel dye, poly(hydroxyethyl methacrylate)-dye copolymers, riboflavin, and copolymers, derivatives, and mixtures thereof. The application method classification of dyes uses the terms reactive dyes, direct dyes, mordant dyes, pigment dyes, anionic dyes, ingrain dyes, vat dyes, sulfur dyes, disperse dyes, basic dyes, cationic dyes, solvent dyes, and acid dyes.

A “dye carrier”, or dyeing accelerant, enables dye penetration into fibers, particularly polyester, cellulose acetate, polyamide, polyacrylic, and cellulose triacetate fibers. The penetration of the dye carrier into the fiber lowers the glass-transition temperature, T_{g}, of the fiber and allows a water-insoluble dye to be taken into the fiber. Most dye carriers are aromatic compounds. Examples of dye carriers include phenolics (e.g., o-phenylphenol, p-phenylphenol, and methyl crestotinate), chlorinated aromatics (e.g., o-dichlorobenzene, and 1,3,5-trichlorobenzene), aromatic hydrocarbons and ethers (e.g., biphenyl, methylbiphenyl, diphenyl oxide, 1-methylnaphthalene, and 2-methylnaphthalene), aromatic esters (e.g., methyl benzoate, butyl benzoate, and benzyl benzoate), and phthalates (e.g., dimethyl phthalate, diethyl phthalate, diallyl phthalate, and dimethyl terephthalate).

A “dye intermediate” refers to a dye precursor or intermediate. A dye intermediate, as used herein, includes both primary intermediates and dye intermediates. Dye intermediates are generally divided into carbocycles, such as benzene, naphthalene, sulfonic acid, diazo-1,2,4-acid, anthraquinone, phenol, aminothiazole nitrate, aryldiazonium salts, arylalkylsulfones, toluene, anisole, aniline, anilide, and chrysazin, and heterocycles, such as pyrazolones, pyridines, indoles, triazoles, aminothiazoles, aminobenzothiazoles, benzoisothiazoles, triazines, and thiopenes.

An “ink” refers to a liquid or paste containing various pigments and/or dyes used for coloring a surface to produce an image, text, or design. Liquid ink is commonly used for drawing and/or writing with a pen, brush or quill. Paste inks are generally thicker than liquid inks. Paste inks are used extensively in letterpress and lithographic printing.

“Insoluble” refers to materials that are intended to be and/or remain as solids in water and are able to be retained in a device, such as a column, or be readily recovered using physical means, such as filtration. Insoluble materials should be capable of prolonged exposure to water, over weeks or months, with little (that is, less than about 5%) loss of mass.

“Oxyanion” or oxoanion is a chemical compound with the generic formula AₓO_{y}^{z-} (where A represents a chemical element other than oxygen and O represents an oxygen atom). In oxyanions, “A” represents metal, metalloid, and/or a nonmetal (e.g., B, P, S, N, and Se) atom. Examples for metal-based oxyanions include chromate, tungstate, molybdate, aluminates, zirconate, etc. Examples of metalloid-based oxyanions include arsenate, arsenite, antimonate, germanate, silicate, etc.

“Particle” refers to a solid, liquid or microencapsulated liquid having a size that ranges from less than one micron to greater than 100 microns, with no limitation in shape.

“Precipitation” refers not only to the removal of a species from a fluid (that from a gaseous or liquid phase) in the form of an insoluble species but also to the immobilization of the species on or in an insoluble particle. For example, “precipitation” includes adsorption and/or absorption.

A “pigment” is a synthetic or natural (biological or mineral) material that changes the color of reflected or transmitted light as the result of wavelength-selective absorption. This physical process differs from fluorescence, phosphorescence, and other forms of luminescence, in which a material emits light. The pigment may comprise inorganic and/or organic materials. Inorganic pigments include elements, their oxides, mixed oxides, sulfides, chromates, silicates, phosphates, and carbonates. Examples of inorganic pigments, include cadmium pigments, carbon pigments (e.g., carbon black), chromium pigments (e.g., chromium hydroxide green and chromium oxide green), cobalt pigments, copper pigments (e.g., chlorophyllin and potassium sodium copper chlorophyllin), pyrogallol, pyrophyllite, silver, iron oxide pigments, clay earth pigments, lead pigments (e.g., lead acetate), mercury pigments, titanium pigments (e.g., titanium dioxide), ultramarine pigments, aluminum pigments (e.g., alumina, aluminum oxide, and aluminum powder), bismuth pigments (e.g., bismuth vanadate, bismuth citrate and bismuth oxychloride), bronze powder, calcium carbonate, chromium-cobalt-aluminum oxide, cyanide iron pigments (e.g., ferric ammonium ferrocyanide, ferric and ferrocyanide), manganese violet, mica, zinc pigments (e.g., zinc oxide, zinc sulfide, and zinc sulfate), spinels, rutiles, zirconium pigments (e.g., zirconium oxide and zircon), tin pigments (e.g., cassiterite), cadmium pigments, lead chromate pigments, luminescent pigments, lithopone (which is a mixture of zinc sulfide and barium sulfate), metal effect pigments, nacreous pigments, transparent pigments, and mixtures thereof. Examples of synthetic organic pigments include ferric ammonium citrate, ferrous gluconate, dihydroxyacetone, guaiazulene, and mixtures thereof. Examples of organic pigments from biological sources include alizarin, alizarin crimson, gamboge, cochineal red, betacyanins, betataxanthins, anthocyanin, logwood extract, pearl essence, paprika, paprika oleoresins, saffron, turmeric, turmeric oleoresin, rose madder, indigo, Indian yellow, tagetes meal and extract, Tyrian purple, dried algae meal, henna, fruit juice, vegetable juice, toasted partially defatted cooked cottonseed flour, quinacridone, magenta, phthalo green, phthalo blue, copper phthalocyanine, indanthone, triarylcarbonium sulfonate, triarylcarbonium PTMA salt, triaryl carbonium Ba salt, triarylcarbonium chloride, polychloro copper phthalocyanine, polybromochlor copper phthalocyanine, monoazo, disazo pyrazolone, monoazo benzimid-azolone, perinone, naphthol AS, beta-naphthol red, naphthol AS, disazo pyrazolone, BONA, beta naphthol, triarylcarbonium PTMA salt, disazo condensation, anthraquinone, perylene, diketopyrrolopyrrole, dioxazine, diarylide, isoindolinone, quinophthalone, isoindoline, monoazo benzimidazolone, monoazo pyrazolone, disazo, benzimidazolones, diarylide yellow dintraniline orange, pyrazolone orange, para red, lithol, azo condensation, lake, diaryl pyrrolopyrrole, thioindigo, aminoanthraquinone, dioxazine, isoindolinone, isoindoline, and quinphthalone pigments, and mixtures thereof. Pigments can contain only one compound, such as single metal oxides, or multiple compounds. Inclusion pigments, encapsulated pigments, and lithopones are examples of multi-compound pigments. Typically, a pigment is a solid insoluble powder or particle having a mean particle size ranging from about 0.1 to about 0.3 μm, which is dispersed in a liquid. The liquid may comprise a liquid resin, a solvent or both. Pigment-containing compositions can include extenders and opacifiers.

A “rare earth” refers to one or more of yttrium, scandium, lanthanum, cerium, praseodymium, neodymium, samarium, europium, gadolinium, terbium, dysprosium, holmium erbium, thulium, ytterbium, and lutetium. As will be appreciated, lanthanum, cerium, praseodymium, neodymium, samarium, europium, gadolinium, terbium, dysprosium, holmium erbium, thulium, ytterbium, and lutetium are known as lanthanoids. The rare earth composition may be water-soluble or water-insoluble. Preferably, the rare earth composition comprises a rare earth having one of +3 or +4 oxidation state. In one embodiment, the rare earth composition comprises a water-soluble rare earth composition having a +3 oxidation state. Non-limiting examples of suitable water-soluble rare earth compositions are rare earth chlorides, nitrates, sulfates, oxalates, and mixtures thereof. Preferably, the water insoluble rare earth composition comprises a rare earth oxide, such as, but not limited to CeO₂.

A “quinone” refers to any member of a class of cyclic aromatic compounds having a fully configurable cyclic dione structure, derived from aromatic compounds by conversion of an even number of ═CH— group into >C═O groups with any necessary rearrangement of double bonds (including polycyclic and heterocyclic analogues).

“Soluble” refers to materials that readily dissolve in water. For purposes of this invention, it is anticipated that the dissolution of a soluble compound would necessarily occur on a time scale of minutes rather than days. For the compound to be considered to be soluble, it is necessary that the compound have a significantly high solubility product such that upwards of 5 g/L of the compound will be stable in solution.

“Sorb” refers to adsorption and/or absorption.

The preceding is a simplified summary of the invention to provide an understanding of some aspects of the invention. This summary is neither an extensive nor exhaustive overview of the invention and its various embodiments. It is intended neither to identify key or critical elements of the invention nor to delineate the scope of the invention but to present selected concepts of the invention in a simplified form as an introduction to the more detailed description presented below. As will be appreciated, other embodiments of the invention are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present invention(s). These drawings, together with the description, explain the principles of the invention(s). The drawings simply illustrate preferred and alternative examples of how the invention(s) can be made and used and are not to be construed as limiting the invention(s) to only the illustrated and described examples. Further features and advantages will become apparent from the following, more detailed, description of the various embodiments of the invention(s), as illustrated by the drawings referenced below.

FIG. 1 is a flow chart depicting a process according to an embodiment;

FIG. 2A is photograph of Direct Blue 15 dye solution prior to addition of ceria;

FIG. 2B is a photograph of a filtrate of the Direct Blue 15 dye solution after the addition of ceria;

FIG. 3A is photograph of Acid Blue 25 dye solution prior to addition of ceria;

FIG. 3B is a photograph of a filtrate of the Acid Blue 25 dye solution after the addition of ceria;

FIG. 4A is photograph of Acid Blue 80 dye solution prior to addition of ceria;

FIG. 4B is a photograph of a filtrate of the Acid Blue 80 dye solution after the addition of ceria;

FIG. 5A is a photograph of ceria-containing Direct Blue 15 solution 2 minutes after adding ceria to the solution;

FIG. 5B is a photograph of ceria-containing Direct Blue 15 solution 10 minutes after adding ceria to the solution;

FIG. 6A is a photograph of ceria-containing Acid Blue 25 solution 2 minutes after adding ceria to the solution;

FIG. 6B is a photograph of ceria-containing Acid Blue 25 solution 10 minutes after adding ceria to the solution;

FIG. 7A is a photograph of ceria-containing Acid Blue 80 solution 2 minutes after adding ceria to the solution; and

FIG. 7B is a photograph of ceria-containing Acid Blue 80 solution 10 minutes after adding ceria to the solution.

### DETAILED DESCRIPTION

In one aspect, the present invention uses an insoluble or soluble rare earth-containing composition or both to remove selected contaminants of a colorant-containing aqueous solution. The colorant-containing aqueous solution can include in addition to the colorant other materials, such as dye intermediates, dye or pigment carriers, extenders, and opacifiers. In one embodiment, the rare earth composition is contacted with the colorant-containing aqueous solution containing one or more of a dye, dye intermediate, dye carrier, or mixture thereof to substantially remove, deactivate, precipitate, remove, decompose, degrade, decolorize, de-toxify, sorb, and/or render colorless the dye, dye intermediate, dye carrier, or mixture thereof.

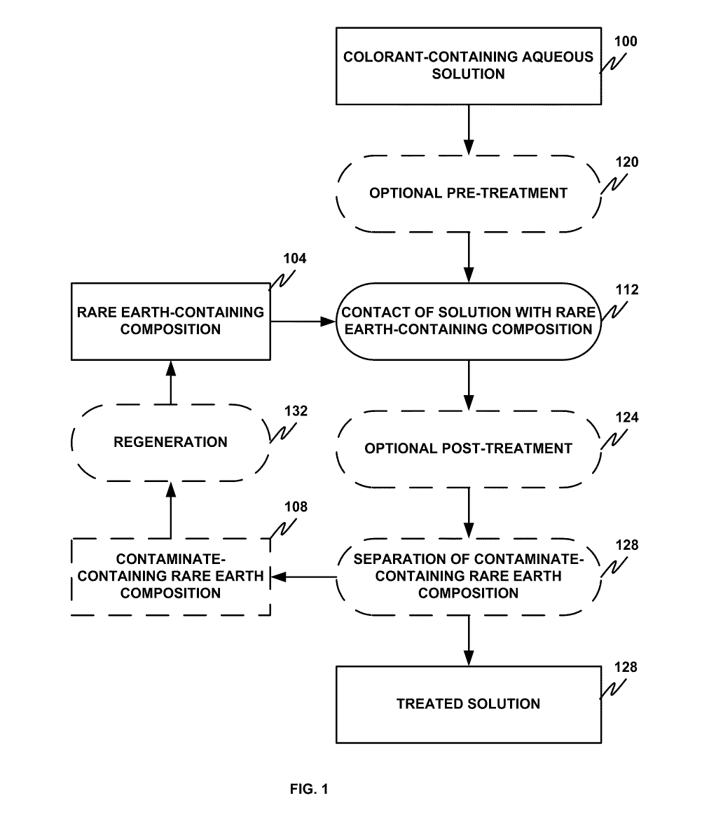

With reference to FIG. 1, the colorant containing aqueous solution 100 can be in any suitable form, whether in the form of a contaminated waste stream, process stream, or natural or man-made body of water. Examples of containing aqueous solutions that can be effectively treated include solutions in potable water systems in wastewater treatment systems, and feed, process, or waste streams in various industrial processes, among others. The described processes, apparatuses, and articles can be used to remove colorants, dye intermediates, and/or dye carriers from solutions having diverse volume and flow rate characteristics and applied in a variety of fixed, mobile, and portable applications. While portions of the disclosure describe the removal of colorants, dye intermediates, and/or dye carriers from water, and particularly potable water streams, preferably by precipitation, such references are illustrative and not to be construed as limiting.

The rare earth-containing composition 104 can comprise a more or more rare earths. The rare earths may be of the same or different valence and/or oxidation states, such as the +3 and +4 oxidation states. The rare earths can be a mixture of different rare earths, such as two or more of yttrium, scandium, cerium, lanthanum, praseodymium, and neodymium.

The rare earth-containing composition may be formulated as water-soluble composition. In one formulation, the rare earth-containing composition is water-soluble and preferably includes one or more rare earths, such as cerium and/or lanthanum, the one or more rare earths having a +3 oxidation state. Non-limiting examples of suitable water soluble rare earth compounds include rare earth halides, rare earth nitrates, rare earth sulfates, rare earth oxalates, rare earth perchlorates and mixtures thereof.

The rare earth can be present in the rare earth-containing composition 104 in the form of one or more of a granule, powder, crystal, crystallite, particle, agglormate, or other particulate, referred to generally herein as a “particulate”. When the rare earth is in the form of a free-flowing powder, the rare earth powder comprises crystals or crystallites. Typically the crystals or crystallites are present as nanocrystals or nanocrystallites. Typically, the rare earth powder has nanocrystallitine domains. The rare earth powder has a mean particle size of at least about 0.5 nm, ranging up to about 1 μm or more. More typically, the rare earth crystals or crystallites have a mean particle size of at least about 1 nm, in some cases greater than about 5 nm, in other cases, at least about 10 nm, and still other cases at least about 25 nm, and in yet still other cases at least about 50 nm. In other embodiments, the particulates are agglomerates having mean particle sizes of at least about 100 nm, specifically at least about 250 nm, more specifically at least about 500 nm, still more specifically at least about 1 μm and yet more specifically at least about 0.5 nm, ranging up to about 1 micron or more.

The rare earth-containing composition 104 may be formulated as agglomerate. In one formulation, the rare earth-containing composition 104 is in the form of free-flowing agglomerates comprising a rare earth powder having nanocrystalline domains and a binder. This agglomerate composition is described in co-pending U.S. application Ser. No. 11/932,702, filed Oct. 31, 2007, which is incorporated in its entirety herein by this reference.

In a preferred agglomerate formulation, the agglomerates include an insoluble rare earth composition, preferably cerium (IV) oxide, cerium (IV) ammonium sulfate, cerium (IV) sulfate, cerium (III) oxide, and mixtures thereof, and a soluble rare earth composition, preferably a cerium (III) salt or oxide (such as cerium (III) carbonate, cerium (III) cerium (III) halides, cerium (III) nitrate, cerium (III) sulfate, cerium (III) oxalates, and mixtures thereof) and/or a lanthanum (III) salt or oxide (such as lanthanum (III) carbonate, lanthanum (III) lanthanum (III) halides, lanthanum (III) nitrate, lanthanum (III) sulfate, lanthanum (III) oxalates, lanthanum (III) oxide, and mixtures thereof).

The binder can include one or more polymers selected from the group consisting of thermosetting polymers, thermoplastic polymers, elastomeric polymers, cellulosic polymers and glasses. Where the binder comprises an ethylene-vinyl copolymer, the insoluble rare earth-containing compound is primarily an anhydrous insoluble rare earth-containing compound.

Suitable thermosetting polymers for the binder include, but are not limited to, polyurethanes, silicones, fluorosilicones, phenolic resins, melamine resins, melamine formaldehyde, and urea formaldehyde. Suitable thermoplastics can include, but are not limited to, nylons and other polyamides, polyethylenes (including LDPE, LLDPE, HDPE, and polyethylene copolymers with other polyolefins), polyvinylchlorides (both plasticized and unplasticized), fluorocarbon resins (such as polytetrafluoroethylene), polystyrenes, polypropylenes, cellulosic resins (such as cellulose acetate butyrates), acrylic resins (such as polyacrylates and polymethylmethacrylates), thermoplastic blends or grafts such as acrylonitrile-butadiene-styrenes or acrylonitrile-styrenes, polycarbonates, polyvinylacetates, ethylene vinyl acetates, polyvinyl alcohols, polyoxymethylene, polyformaldehyde, polyacetals, polyesters (such as polyethylene terephthalate), polyether ether ketone, phenol-formaldehyde resins (such as resols and novolacs) and mixtures thereof. Suitable elasomers can include, but are not limited to, natural and/or synthetic rubbers, like styrene-butadiene rubbers, neoprenes, nitrile rubber, butyl rubber, silicones, polyurethanes, alkylated chlorosulfonated polyethylene, polyolefins, chlorosulfonated polyethylenes, perfluoroelastomers, polychloroprene (neoprene), ethylene-propylene-diene terpolymers, chlorinated polyethylene, fluoroelastomers, and ZALAK™ (Dupont-Dow elastomer). Those of skill in the art will realize that some of the thermoplastics listed above can also be thermosets depending upon the degree of cross-linking, and that some of each may be elastomers depending upon their mechanical properties. The categorization used above is for ease of understanding and should not be regarded as limiting or controlling.

Cellulosic polymers as binders can include naturally occurring cellulose such as cotton, paper and wood and chemical modifications of cellulose. In a specific embodiment, the rare earth-containing compound can be mixed with paper fibers or incorporated directly into paper pulp for forming a paper-based filter comprising the rare earth-containing compound. Preferably, the rare earth containing compound mixed with the paper fibers or incorporated directly into the paper pulp is an insoluble rare earth-containing composition.

Other suitable binders include glass materials such as glass fibers, beads and mats. Glass solids may be mixed with particulates of an insoluble rare earth-containing compound and heated until the solids begin to soften or become tacky so that the insoluble rare earth-containing compound adheres to the glass. Similarly, extruded or spun glass fibers may be coated with particles of the insoluble rare earth-containing compound while the glass is in a molten or partially molten state or with the use of adhesives. Alternatively, the glass composition may be doped with the insoluble rare earth-containing compound during manufacture. Techniques for depositing or adhering insoluble rare earth-containing compounds to a substrate material are described in U.S. Pat. No. 7,252,694 and other references concerning glass polishing. For example, electro-deposition techniques and the use of metal adhesives are described in U.S. Pat. No. 6,319,108 as being useful in the glass polishing art. U.S. Pat. Nos. 6,319,108 and 7,252,694 are incorporated herein by this reference in their entirety.

In some applications, water-soluble glasses such as are described in U.S. Pat. Nos. 5,330,770, 6,143,318 and 6,881,766, may be an appropriate binder. The descriptions of such water-soluble glasses contained in U.S. Pat. Nos. 5,330,770, 6,143,318 and 6,881,766 are incorporated herein by this reference in their entirety. In other applications, materials that swell through fluid absorption including but not limited to polymers such as synthetically produced polyacrylic acids, and polyacrylamides and naturally-occurring organic polymers such as cellulose derivatives may also be used as binders. Biodegradable polymers such as polyethylene glycols, polylactic acids, polyvinylalcohols, co-polylactideglycolides, and the like may also be used as the binder.

The preferred mean, median, or P₉₀ size of the agglomerates depends on the application. In most applications, the agglomerates preferably have a mean, median, or P₉₀ size of at least about 1 μm, more preferably at least about 5 μm, more preferably at least about 10 μm, still more preferably at least about 25 μm. In other applications, the agglomerate has a mean, median, or P₉₀ particle size distribution from about 100 to about 1,000 microns, a mean, median, or P₉₀ particle size distribution from about 200 to about 600 microns, or a mean, median, or P₉₀ particle size distribution from about 300 to about 500 microns. The mean surface area of the agglomerates preferably is at least about 70 m²/g, even more preferably at least about 85 m²/g, even more preferably at least about 115 m²/g, and even more preferably at least about 125 m²/g.

The agglomerates can be formed through one or more of extrusion, molding, calcining, sintering, and compaction. Preferably, the agglomerates include more than 10.01%, even more preferably more than about 75%, and even more preferably from about 80 to about 95% of the rare earth-containing composition, with the balance being primarily the binder. Stated another way, the binder can be less than about 15% by weight of the agglomerate, in some cases less than about 10% by weight, and in still other cases less than about 8% by weight of the agglomerate.

In another formulation, the rare earth-containing composition includes nanocrystalline rare earth particles supported on, coated on, or incorporated into a substrate, such as described in U.S. Pat. No. 6,863,825 to Witham, et al., which is incorporated herein by this reference in its entirety. The nanocrystalline rare earth particles can, for example, be supported or coated on the substrate by a suitable binder. The substrate can be a sintered ceramic, sintered metal, microporous carbon, glass fiber, cellulosic fiber, alumina, gamma-alumina, activated alumina, acidified alumina, metal oxide containing labile anions, crystalline alumino-silicate such as a zeolite, amorphous silica-alumina, ion exchange resin, clay, ferric sulfate, porous ceramic, and the like. The structure of the substrate will vary depending on the application but can include a woven substrate, non-woven substrate, porous membrane, filter, or other fluid permeable structure. Substrates can also include porous and fluid permeable solids having a desired shape and physical dimensions. Such substrates can include mesh, screens, tubes, honeycomb structures, monoliths, and blocks of various shapes, including cylinders and toroids. For example, the rare earth particles can be incorporated into or coated onto a filter block or monolith for use in a filter, such as a cross-flow type filter. The rare earth can also be ionically substituted for cations naturally occurring on the substrate. Ionic substitution is well known in the art.

It should be noted that it is not required to formulate the rare earth-containing composition with neither a binder nor a substrate, though such formulations may be desired depending on the application.

The rare earth composition may be formulated as powder. In such a formulation, the rare earth-containing composition is in the form of a free-flowing, finely divided soluble or insoluble powder, which is contacted with the colorant-containing solution.

The colorant-containing aqueous solution comprises one or more of a colorant, dye intermediate, dye carrier, pigment carrier, extender, and opacifier. In one configuration, a selected contaminant (that is, one of the colorant, dye intermediate, dye carrier, pigment carrier, extender, and opacifier) of the colorant-containing aqueous solution is precipitated, de-toxified, or otherwise removed by the rare earth-containing composition. The colorant can be a pigment, dye, or mixture thereof, with a dye being common. In one configuration, the colorant-containing aqueous solution is rendered substantially colorless by the rare earth-containing composition. While not wishing to be bound by any theory, the colorant-containing aqueous solution is believed to be rendered substantially colorless by precipitation or degradation of the contaminant, preferably the colorant, while the colorant is believed to be rendered substantially colorless by precipitation of the colorant and/or degradation of the chromophore and/or auxochrome of the colorant. Moreover, it is further believed that at least some, or most or all, of the contaminant and/or colorant contained within the aqueous solution is sorbed by the rare earth-containing composition to from a contaminate-containing rare earth composition 108. Typically, the rare earth-containing composition reduces a colorant-emanating color of the aqueous solution (before contact with the rare earth-containing composition) to a level of less than about 200 ppb, to a level of less than about 100 ppb, to a level of less than about 50 ppb, to a level of less than about 10 ppb, to a level of less than about 1 ppb, or to a level of less than about 0.1 ppb.

While not wishing to be bound by any theory, the rare earth containing composition precipitates colorants, dye intermediates, and/or dye carriers having a suitable organic or inorganic functional group or inorganic anion or cation. Suitable organic or inorganic functional groups include electro-negative functional groups, such as halide, ether, carboxy, carboxylic, or carbonyl functional groups or their analogs, as for example where one or more oxygen atom are substituted with a sulfur, nitrogen atom or a combination thereof. Suitable anionic functional groups or inorganic ions include oxyanions of certain metals, metalloids, and nonmetals. The metals and metalloids typically include elements having an atomic number selected from the group of consisting of atomic numbers 5, 9, 13, 14, 22 to 25, 31, 32, 33, 34, 35, 40 to 42, 44, 45, 49 to 53, 72 to 75, 77, 78, 80, 81, 82, 83, 85, 92, 94, 95, and 96 and even more preferably from the group consisting of atomic numbers 5, 13, 14, 22 to 25, 31, 32, 33, 34, 40 to 42, 44, 45, 49 to 52, 72 to 75, 77, 78, 80, 81, 82, 83, 92, 94, 95, and 96. These atomic numbers include the elements of arsenic, aluminum, astatine, bromine, boron, fluorine, iodine, silicon, titanium, vanadium, chromium, manganese, gallium, thallium, germanium, selenium, mercury, zirconium, niobium, molybdenum, ruthenium, rhodium, indium, tin, antimony, tellurium, hafnium, tantalum, tungsten, rhenium, iridium, platinum, lead, uranium, plutonium, americium, curium, and bismuth. Examples of suitable nonmetal oxyanions reactive with the rare earth-containing composition include carbonates, phosphates, organophosphates, phosphites, sulfates, sulfites, nitrates, and nitrites.

The contaminate-containing rare earth composition 108 includes the selected contaminant or a derivative thereof sorbed or otherwise attached to the rare earth-containing composition 104. For example, the dye or a derivative of the dye forms an insoluble precipitate with the rare earth in the rare earth-containing composition 104. The precipitate can then be removed from the aqueous solution.

Using different rare earths and/or different oxidation states of rare earths can provide a number of beneficial results. For example, different rare earths or different oxidation states of rare earths can be effective in removing different contaminants from the colorant-containing aqueous solution. The different contaminants include different ones of dyes, pigments, dye intermediates, dye carriers, pigment carriers, extenders, and/or opacifiers. The use of different rare earths and/or different oxidation states can also cause one or more of the rare earth compounds in the rare earth-containing composition to be insoluble in the aqueous solution and one or more others of the rare earth compounds in the rare earth-containing composition to be soluble in the aqueous solution. Thus, when the insoluble and soluble rare earth compounds target a common contaminant, different removal mechanisms can be used to provide better contaminant removal. Solubility can provide better dispersion of the rare earth compound throughout the aqueous solution while insolubility requires particle-to-particle interaction to occur for effective removal.

In step 112, the rare earth-containing composition 104 is contacted with the colorant-containing aqueous solution to produce a treated solution 116. Contact between the colorant-containing aqueous solution and the rare earth-containing composition can be achieved by flowing the solution 100 through the composition 104 or by adding the composition 104 to the solution 100, with or without mixing or agitation. Contact with the rare earth-containing composition 104 is commonly sufficient to remove, deactivate, detoxify, precipitate, and/or render colorless the selected contaminants in the solution 100 and other types of treatment to effect such removal are optional.

In some embodiments, the rare earth-containing composition 104 is distributed over the surface of the aqueous solution 100 and allowed to settle, under the influence of gravity, through the solution. Such an application is particularly useful for treating selected contaminants in solutions found in evaporation tanks, municipal water treatment systems, fountains, ponds, lakes and other natural or man-made bodies of water. In such embodiments, it is preferred, but not required, that the contaminated rare earth-containing composition be filtered or otherwise separated from the solution for disposal or regeneration and re-use.

In other embodiments, the rare earth-containing composition 104 can be introduced into the flow of the aqueous solution 100, such as through a conduit, pipe or the like.

In other embodiments, the rare earth-containing composition 104 is disposed in a container, and the solution 100 directed to flow through the composition 104. The aqueous solution can flow through the composition under the influence of gravity, pressure or other means and with or without agitation or mixing. In still other embodiments, the container comprises a fluid permeable outer wall encapsulating the rare earth-containing composition so that the solution has multiple flow paths through the rare earth-containing composition when submerged. Various fittings, connections, pumps, valves, manifolds and the like can be used to control the flow of the rare earth-containing solution through the composition in a given container.

The aqueous solution 100 contacts the rare earth-containing composition 104 at a temperature above the triple point for the solution. In some cases, the solution contacts the rare earth-containing composition at a temperature less than about 100° C. and in other cases, contact occurs at a temperature above about 100° C., but at a pressure sufficient to maintain at least a portion of the aqueous solution in a liquid phase. The rare earth-containing composition is effective at removing and detoxifying selected contaminants at room temperatures. In other cases, the aqueous solution contacts the rare earth-containing composition under supercritical conditions of temperature and pressure for the aqueous solution.

The pressure at which the aqueous solution 100 contacts the rare earth-containing composition 104 can vary considerably depending on the application. For smaller volume applications where the contact is to occur within a smaller diameter column at a flow rates less than about 1.5 gpm, the pressure can range from 0 up to about 6.0 psig. In applications where larger containers and higher flow rates are employed, higher pressures may be required.

In optional pretreatment step 120, the aqueous solution is subjected to one or more treatment steps to render the colorant-containing aqueous solution 100 suitable for treatment by the rare earth-containing composition 104. In one process configuration, optional pretreatment includes removing contaminants or other solution components that may interfere with the removal of the selected contaminant by the rare earth-containing composition 104. A common type of interferor competes with the selected contaminant for sites in and/or the rare earth-containing composition. Such interferors, for example, include fluorides, phosphates, carbonates, silicates, and vanadium oxides. Removal of the interferor can be effected by any suitable technique, including precipitation by a non-rare-earth sorbent and/or pH adjustment, ion exchange, membrane filtration, precipitation, a complexing agent, and the like. In this application, the concentration of interferors is maintained preferably at a concentration of no more than about 300 ppm/interferor species and even more preferably no more than about 10 ppm/interferor species.

In one process configuration where the selected contaminant is a pigment, the pigment is substantially dissolved, dispersed or solubilized in the aqueous solution by suitable techniques, including pH adjustment by a suitable acid or base. In one process configuration where the selected contaminant is a colorant, dye intermediate, dye carrier, pigment carrier, extender, or opacifier, the selected contaminant is decomposed by a chemically (such as by an oxidant or reductant), thermally, and/or optically to provide derivatives more suitable for removal by the rare earth-containing composition.

Following the contacting step 112, the aqueous solution can be further optionally treated by one or more post-treatment steps 124. Such post-treatment steps 124 include, for example, removal of other contaminants by other techniques, such as by precipitation by a non-rare-earth sorbent, ion exchange, membrane filtration, a pH adjustment, precipitation, complexation with a complexing or chelating agent, and the like.

Following the optional post-treatment step(s) 124 or contacting step 112, the contaminate-containing rare earth composition 108 is, in optional step 128, separated from the treated solution 116 substantially depleted of selected contaminants. The composition can be separated from the solution by conventional liquid-solid separation techniques including, but not limited to, the use of filters, membranes, settling tanks, centrifuges, cyclones or the like. The separated solution, substantially depleted of selected contaminants can then be directed to further processing, storage or use.

In one process configuration, the rare earth-containing composition is introduced into the solution colorant-containing 100 upstream of a filter, where the contaminated rare earth-containing composition is separated and recovered from the solution. A particular example of such an embodiment can be found in municipal water treatment operations where the rare earth-containing composition is injected or otherwise introduced into the water treatment system upstream of a particulate filter bed.

Where an apparatus is employed to contact the rare earth-containing composition 104 with the colorant-containing solution 100, the apparatus can include a filter for separating the treated solution 116 from the contaminate-containing rare earth composition 108. The filter can encapsulate the rare earth-containing composition or be disposed downstream of the composition. Moreover, the filter can be a feature of the container for preventing the rare earth-containing composition from flowing out of the container or be a feature of the apparatus disposed downstream of the container. The filter can include woven and non-woven fabrics, mesh, as well as fibers or particulates that are disposed in a mat, bed or layer that provides a fluid permeable barrier to the rare earth-containing composition. Where the rare earth-containing composition is disposed in a fixed bed, a suitable filter can include a layer of diatomaceous earth disposed downstream of the rare earth-containing composition within the container.

The separated, precipitated product or composition 108 can be subjected to one or more regeneration mechanisms in step 132 to remove, from the contaminate-containing rare earth composition, substantially all of the sorbed contaminant and permit the regenerated rare earth-containing composition to be recycled or reused in the contacting step 112. Moreover, it may be desirable to sterilize or regenerate the contaminate containing rare earth composition 108 prior to contacting the aqueous solution to remove any further contaminants that may be present before re-use or disposing of the composition. Sterilization or regeneration processes can include thermal processes wherein the contaminate-containing rare earth composition 108 is exposed to elevated temperatures or pressures or both to degrade or decompose the sorbed contaminant, radiation sterilization wherein the contaminated rare earth-containing composition 108 is subjected to elevated radiation levels, including processes using ultraviolet, infrared, microwave, and ionizing radiation, to degrade or decompose the sorbed contaminant, biological decomposition, wherein the contaminant is decomposed by microbes, and chemical sterilization, wherein the contaminated rare earth-containing composition 108 is exposed to elevated levels of oxidants or reductants or other chemical species to degrade or decompose the sorbed contaminant or replace the sorbed contaminant with another species.

Biological decomposition can be effected by aerobes and anaerobes, such as bacteria and fungi, that are biologically active in the presence of rare earths. In the presence of specific oxygen-catalyzed enzymes called azo reductases, some aerobic bacteria, such as the Pseudomonas species strains K22 and KF46 and Pagmentiphaga kullae K24, reduce azo compounds and produce aromatic amines. Clostridium, Salmonella, Bacillus, Eubacterium, Excherichia coli are believed to reduce the dyes ingested through food, drugs, and cosmetics. In the presence of fungal strains, such as Bjerkandera adusta, Trametes versicolor, Polyporus pinistus, Myceliophthora thermophilia, Pyricularia oryzae, and Phanerochaete chrysosporium, can degrade one or more of azo dyes due to the formation of exoenzymes, such as peroxidases and phenoloxidases, anthraquinone dyes, and indigoid dyes. Under anaerobic conditions, a low redox potential (less than or equal to about 50 mV) can be achieved, which enables decolorization of dyes, which is believed to decolorize dyes by a combination of biological and chemical mechanisms. Azo dye reduction is believed to mostly occur by extracellular or membrane-bond enzymes. The biological contribution can be divided in specialized enzymes called azo reductases, which are present in bacteria, such as Sphingomonas xenophaga strain BN6, that are able to grow using only azo dye as a carbon and energy source. The chemical contribution to the reductive decolorization of azo dyes under anaerobic conditions may involve biogenic reductants, like sulfide, cysteine, ascorbate, or divalent iron. Redox mediators, or compounds that accelerate the electron transfer mechanism from a primary electron donor to a terminal electron acceptor, are believed to increase the reaction rates for reductive decolorization. Anaerobic granular sludge has been found to be effective in decolorizing azo dyes.

Chemical species that may be used in chemical sterilization to degrade or decompose colorants include oxidants, such as ozone, hydrogen peroxide and other inorganic peroxides, nitric acid and nitrates, chlorite, chlorate, perchlorate and other analogous halogen compounds, hypochlorite and other hypohalite compounds (such as bleach), iodine and other halogens other than fluoride, nitrous oxide (N₂O), silver oxide, hexavalent chromium compounds persulfuric acid, sulfoxides, sulfuric acid, Tollens' reagent, 2,2′-dipyridyldisulfide aka DPS, and reductants, such as lithium aluminium hydride (LiAlH₄), nascent hydrogen, sodium amalgam, sodium borohydride (NaBH₄), compounds containing the Sn²⁺ ion (such as tin (II) chloride), sulfite compounds, hydrazine (Wolff-Kishner reduction), zinc-mercury amalgam (Zn(Hg)) (Clemmensen reduction), diisobutylaluminium hydride (DIBAH), Lindlar catalyst, formic acid (HCOOH), dithiothreitol (DTT), and compounds containing the Fe²⁺ ion (such as iron (II) sulfate).

Combinations of the above processes can be used. An example is the hydrogen peroxide/UV process which forms HO radicals. Such sterilization processes may be used on a sporadic or continuous basis while the composition is in use.

The process can optionally include the step of sensing (not shown) the solution depleted of the selected contaminants so as to determine or calculate when it is appropriate to replace the rare earth-containing composition. Sensing of the solution can be achieved through conventional means such as tagging and detecting the contaminants in the aqueous solution using fluorescent or radioactive materials, measuring flow rates, temperatures, pressures, sensing for the presence of fines, colorimetry or colorimetric analysis, photometry or photometric analysis, and sampling and conducting arrays. Colorimetry measures the color and/or color intensity, such as for a selected wavelength color range, of the treated solution. A colorimeter, for example, analyzes or quantifies color commonly by measuring a given color in terms of a standard color, a scale of colors, certain primary colors, or other spectroscopic or visual standard. Colorimeters can also measure the concentration of a known constituent of a solution by comparison with colors of standard solutions of that constituent. A photometer is normally a type of diagnostic device that measures optical changes and/or status to measure units. For example, a photometer can measure the properties of light (especially luminous intensity) and compare the determined properties against a defined or predetermined set of properties indicating a desired degree of dye or pigment contaminant removal. In this manner, the degree of dye or pigment contaminant removal can be analyzed.

Where it is desirable to regenerate an agglomerated rare earth-containing composition through sterilization, the selected binder or substrate material should be stable under sterilization conditions and should be otherwise compatible with the sterilization method. Specific non-limiting examples of binders that are suitable for sterilization methods that involve exposure to high temperatures include cellulose nitrate, polyethersulfone, nylon, polypropylene, polytetrafluoroethylene, and mixed cellulose esters. Compositions prepared with these binders can be autoclaved when the prepared according to known standards. Desirably, the agglomerated rare earth-containing composition should be stable to steam sterilization or autoclaving as well as to chemical sterilization through contact with oxidative or reductive chemical species, as a combination of sterilization methods may be required for efficient and effective regeneration. In an embodiment where sterilization includes the electrochemical generation of an oxidative or reductive chemical species, the electrical potential necessary to generate said species can be attained by using the composition as one of the electrodes. For example, an agglomerated rare earth-containing composition that contains a normally insulative polymeric binder can be rendered conductive through the inclusion of a sufficiently high level of conductive particles such as granular activated carbon, carbon black, or metallic particles. Alternatively, if the desired level of carbon or other particles is not sufficiently high to render an otherwise insulative polymer conductive, an intrinsically conductive polymer may included in the binder material. Various glasses such as microporous glass beads and fibers are particularly suited for use as a substrate or binder where the agglomerated rare earth-containing composition is to be periodically regenerated.

### Experimental

The following examples are provided to illustrate certain embodiments of the invention and are not to be construed as limitations on the invention, as set forth in the appended claims. All parts and percentages are by weight unless otherwise specified.

In a first example, twenty 3.6 g packets of cherry Kool-Aid™ unsweetened soft drink mix (containing Red 40 (as azo dye having the composition2-naphthalenesulfonic acid, 6-hydroxy-5-((2-methoxy-5-methyl-4-sulfophenyl)azo) disodium salt, and disodium 6-hydroxy-5-((2-methoxy-5-methyl-4-sulfophenyl)azo)-2-naphthalenesulfonate) and Blue 1 (a disodium salt having the formula C₃₇H₃₄N₂Na₂O₉S₃) dyes) were added to and mixed with five gallons of water. For use in the first test, a column setup was configured such that the dyed water stream enters and passes through a fixed bed of insoluble cerium (IV) oxide to form a treated solution. The dyed, colored water was pumped through the column setup. The treated solution was clear of any dyes, and at the top of the bed there was a concentrated band of color, which appeared to be the Red 40 and Blue 1 dyes.

In a second example, cherry Kool-Aid™ unsweetened soft drink mix (containing Red 40 and Blue 1 dyes) was dissolved in water, and the mixture stirred in a beaker. Insoluble cerium (IV) oxide was added and kept suspended in the solution by stirring. When stirring ceased, the cerium oxide settled, leaving behind clear, or colorless, water. This example is intended to replicate water treatment by a continuous stirred tank reactor (CSTR).

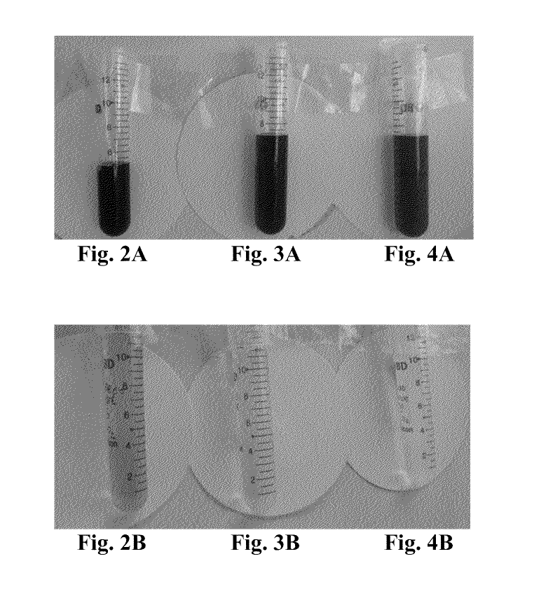

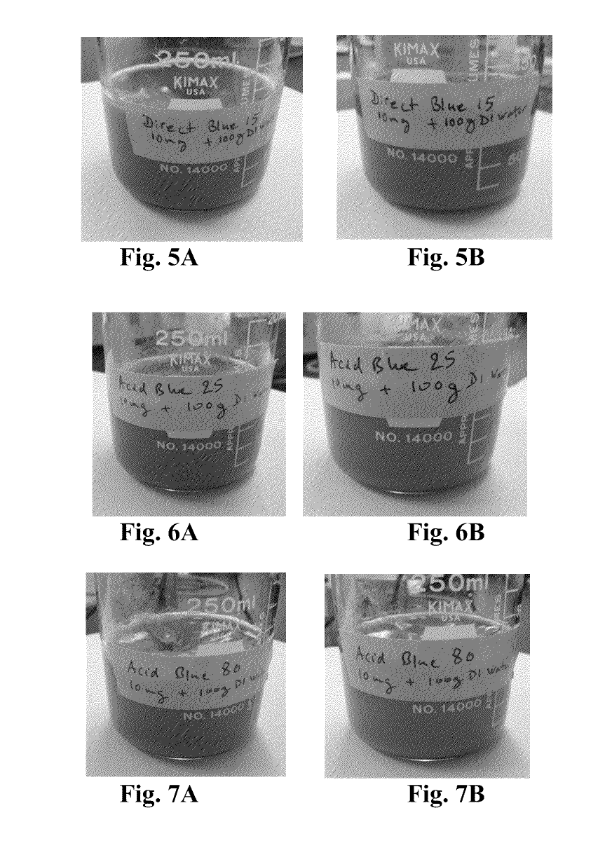

In a third example, 10.6 mg of Direct Blue 15 (C₃₄H₂₄N₆Na₄O₁₆S₄, from Sigma-Aldrich) was dissolved in 100.5 g of de-ionized water. The Direct Blue 15 solution (FIG. 2A) was stirred for 5 min. using a magnetic stir bar before adding 5.0012 g of high surface area ceria (CeO₂). The ceria-containing Direct Blue 15 solution was stirred. The ceria-containing Direct Blue 15 solution 2 min and 10 min after adding the ceria are, respectively, showed in FIGS. 5A and 5B. After stirring for 10 min, a filtrate was extracted using a 0.2 μm syringe filter. The filtrate was clear and substantially colorless, having a slightly visible blue tint (FIG. 2B).

In a fourth example, 9.8 mg of Acid Blue 25 (45% dye content, C₂₀H₁₃N₂NaO₅S, from Sigma-Aldrich) was dissolved in 100.3 g of de-ionized water. The Acid Blue 25 solution (FIG. 3A) was stirred for 5 min. using a magnetic stir bar before adding 5.0015 g of high surface area ceria (CeO₂). The ceria-containing Acid Blue 25 solution was stirred. The ceria-containing Acid Blue 25 solution 2 min and 10 min after adding the ceria are, respectively, showed in FIGS. 6A and 6B. After stirring for 10 min, a filtrate was extracted using a 0.2 μm syringe filter. The filtrate was clear and substantially colorless, and lacked any visible tint (FIG. 3B).

In a fifth example, 9.9 mg of Acid Blue 80 (45% dye content, C₃₂H₂₈N₂Na₂O₈S₂, from Sigma-Aldrich) was dissolved in 100.05 g of de-ionized water. The Acid Blue 80 solution (FIG. 4A) was stirred for 5 min. using a magnetic stir bar before adding 5.0012 g of high surface area ceria (CeO₂). The ceria-containing Acid Blue 80 solution was stirred. The ceria-containing Acid Blue 80 solution 2 min and 10 min after adding the ceria are, respectively, showed in FIGS. 7A and 7B. After stirring for 10 min, a filtrate was extracted using a 0.2 μm syringe filter. The filtrate was clear and substantially colorless, and lacked any visible tint (FIG. 4B).

Based on these experiments and while not wishing to be bound by any theory, the dyes are believed to sorb or otherwise react with the cerium (IV) oxide.

A number of variations and modifications of the invention can be used. It would be possible to provide for some features of the invention without providing others.

The present invention, in various embodiments, configurations, or aspects, includes contaminants, methods, processes, systems and/or apparatus substantially as depicted and described herein, including various embodiments, configurations, aspects, sub-combinations, and subsets thereof. Those of skill in the art will understand how to make and use the present invention after understanding the present disclosure. The present invention, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease and\or reducing cost of implementation.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments, configurations, or aspects for the purpose of streamlining the disclosure. The features of the embodiments, configurations, or aspects of the invention may be combined in alternate embodiments, configurations, or aspects other than those discussed above. This method of disclosure is not to be interpreted as reflecting an intention that the claimed invention requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the invention.

Moreover, though the description of the invention has included description of one or more embodiments, configurations, or aspects and certain variations and modifications, other variations, combinations, and modifications are within the scope of the invention, e.g., as may be within the skill and knowledge of those in the art, after understanding the present disclosure. It is intended to obtain rights which include alternative embodiments, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method, comprising:
contacting a colorant-containing aqueous solution with a rare earth-containing composition to form a treated solution substantially depleted of the colorant.

2. The method of claim 1, wherein the colorant is a dye.

3. The method of claim 2, wherein the dye comprises a chromophore that is at least one of: azo, carbonyl, methane, nitro, hydrazo, anthraquinone, methine, styryl, methane, cyanine, thiazine, and quinone.

4. The method of claim 2, wherein the dye is at least one of an indole, pyrazolone, pyridone, phenol, anthraquinone benzodifuranone indigoid, polymethine, styryl, carbonium, triphenylmethane, diphenylmethane, thiazine, triphendioxazine, phthalocyanine quinophthalone, nitrodiphenylamine, naphthol, stilbene, formazan, hydrazone, azine, xanthene, triarylmethane, azine, acridine, oxazine, pyrazole, pyrazoline, pyrazalone, coumarin, naphthalimide, carotenoid, flavonol, flavone, chroman, aniline black, quinacridone, formazan, triphendioxazine, thiazine, ketone amine, caramel poly(hydroxyethyl methacrylate) copolymers, riboflavin, and derivatives and mixtures thereof.

5. The method of claim 2, wherein the dye is one of a reactive dye, direct dye, mordant dye, pigment dye, anionic dye, ingrain dye, vat dye, sulfur dye, disperse dye, basic dye, cationic dye, solvent dye, and acid dye.

6. The method of claim 2, wherein the colorant-containing solution comprises a dye intermediate and wherein the dye intermediate is precipitated by the rare earth-containing composition.

7. The method of claim 2, wherein the colorant-containing solution comprises a dye carrier and wherein the dye carrier is precipitated by the rare earth-containing composition.

8. The method of claim 1, wherein the colorant is a pigment.

9. The method of claim 8, wherein the pigment is at least one of a cadmium, carbon, chromium, cobalt, copper, pyrogallol, pyrophyllite, silver, iron oxide, clay earth, lead, mercury, titanium, ultramarine, aluminum, bismuth, bronze, calcium carbonate, chromium-cobalt-aluminum oxide, cyanide iron, manganese violet, mica, zinc, spinel, rutile, zirconium, tin, cadmium, lead chromate, luminescent, metal effect, nacreous, transparent, ferric ammonium citrate, ferrous gluconate, dihydroxyacetone, guaiazulene, alizarin, alizarin crimson, gamboge, cochineal red, betacyanins, betataxanthins, anthocyanin, logwood extract, pearl essence, paprika, paprika oleoresins, saffron, turmeric, turmeric oleoresin, rose madder, indigo, Indian yellow, tagetes meal and extract, Tyrian purple, dried algae meal, henna, quinacridone, magenta, phthalo green, phthalo blue, copper phthalocyanine, indanthone, triarylcarbonium sulfonate, triarylcarbonium PTMA salt, triaryl carbonium Ba salt, triarylcarbonium chloride, polychloro copper phthalocyanine, polybromochlor copper phthalocyanine, azo, pyrazolone, monoazo benzimid-azolone, perinone, naphthol AS, beta-naphthol red, naphthol AS, disazo pyrazolone, BONA, beta naphthol, triarylcarbonium PTMA salt, disazo condensation, anthraquinone, perylene, diketopyrrolopyrrole, dioxazine, diarylide, isoindolinone, quinophthalone, isoindoline, monoazo benzimidazolone, monoazo pyrazolone, disazo, benzimidazolones, diarylide yellow dintraniline orange, pyrazolone orange, para red, lithol, azo condensation, lake, diaryl pyrrolopyrrole, thioindigo, aminoanthraquinone, dioxazine, isoindolinone, isoindoline, and quinphthalone pigments, and mixtures thereof.

10. The method of claim 9, further comprising before the contacting step:
at least one of adding a solvent to and adjusting a pH of the colorant-containing solution to dissolve substantially the pigment.

11. The method of claim 9, wherein the rare earth-containing composition precipitates a pigment carrier.

12. The method of claim 9, wherein the rare earth-containing composition precipitates an extender.

13. The method of claim 9, wherein the rare earth-containing composition precipitates an opacifier.

14. The method of claim 1, wherein the colorant-containing aqueous solution has a color and wherein, after the contacting step, the color is substantially removed.

15. The method of claim 1, wherein the rare earth-containing composition comprises a plurality of cerium, lanthanum, praseodymium, and neodymium.

16. The method of claim 1, wherein the rare earth-containing composition comprises plurality rare earth oxidation states.

17. The method of claim 1, wherein the colorant comprises an electro-negative functional group that is one of a halide, ether, carboxy, carboxylic, and carbonyl.

18. The method of claim 1, wherein the colorant comprises an oxyanion of a metal, metalloid, and/or nonmetal.

19. A composition, comprising:
a rare earth; and
a colorant sorbed on the rare earth.

20. The composition of claim 19, wherein the rare earth comprises a plurality of cerium, lanthanum, praseodymium, and neodymium.

21. The composition of claim 19, wherein the colorant is a dye.

22. The composition of claim 21, wherein the dye comprises a chromophore that is at least one of: azo, carbonyl, methane, nitro, hydrazo, anthraquinone, methine, styryl, methane, cyanine, thiazine, and quinone.

23. The composition of claim 21, wherein the dye is at least one of an indole, pyrazolone, pyridone, phenol, anthraquinone benzodifuranone indigoid, polymethine, styryl, carbonium, triphenylmethane, diphenylmethane, thiazine, triphendioxazine, phthalocyanine quinophthalone, nitrodiphenylamine, naphthol, stilbene, formazan, hydrazone, azine, xanthene, triarylmethane, azine, acridine, oxazine, pyrazole, pyrazoline, pyrazalone, coumarin, naphthalimide, carotenoid, flavonol, flavone, chroman, aniline black, quinacridone, formazan, triphendioxazine, thiazine, ketone amine, caramel poly(hydroxyethyl methacrylate) copolymers, riboflavin, and derivatives and mixtures thereof.

24. The composition of claim 21, wherein the dye is one of a reactive dye, direct dye, mordant dye, pigment dye, anionic dye, ingrain dye, vat dye, sulfur dye, disperse dye, basic dye, cationic dye, solvent dye, and acid dye.

25. The composition of claim 21, wherein the colorant comprises a dye intermediate.

26. The composition of claim 21, wherein the colorant comprises a dye carrier.

27. The composition of claim 19, wherein the colorant is a pigment.

28. The composition of claim 27, wherein the pigment is at least one of a cadmium, carbon, chromium, cobalt, copper, pyrogallol, pyrophyllite, silver, iron oxide, clay earth, lead, mercury, titanium, ultramarine, aluminum, bismuth, bronze, calcium carbonate, chromium-cobalt-aluminum oxide, cyanide iron, manganese violet, mica, zinc, spinel, rutile, zirconium, tin, cadmium, lead chromate, luminescent, metal effect, nacreous, transparent, ferric ammonium citrate, ferrous gluconate, dihydroxyacetone, guaiazulene, alizarin, alizarin crimson, gamboge, cochineal red, betacyanins, betataxanthins, anthocyanin, logwood extract, pearl essence, paprika, paprika oleoresins, saffron, turmeric, turmeric oleoresin, rose madder, indigo, Indian yellow, tagetes meal and extract, Tyrian purple, dried algae meal, henna, quinacridone, magenta, phthalo green, phthalo blue, copper phthalocyanine, indanthone, triarylcarbonium sulfonate, triarylcarbonium PTMA salt, triaryl carbonium Ba salt, triarylcarbonium chloride, polychloro copper phthalocyanine, polybromochlor copper phthalocyanine, azo, pyrazolone, monoazo benzimid-azolone, perinone, naphthol AS, beta-naphthol red, naphthol AS, disazo pyrazolone, BONA, beta naphthol, triarylcarbonium PTMA salt, disazo condensation, anthraquinone, perylene, diketopyrrolopyrrole, dioxazine, diarylide, isoindolinone, quinophthalone, isoindoline, monoazo benzimidazolone, monoazo pyrazolone, disazo, benzimidazolones, diarylide yellow dintraniline orange, pyrazolone orange, para red, lithol, azo condensation, lake, diaryl pyrrolopyrrole, thioindigo, aminoanthraquinone, dioxazine, isoindolinone, isoindoline, and quinphthalone pigments, and mixtures thereof.

29. The composition of claim 27, further comprising a polymeric binder and rare earth nanocrystals.

30. A system, comprising:
an inlet for a colorant-containing aqueous solution;
a rare earth-containing composition in a colorant removal zone; and
an outlet for a treated aqueous solution substantially depleted of the colorant.

31. The system of claim 30, wherein the rare earth-containing composition comprises a plurality of cerium, lanthanum, praseodymium, and neodymium.

32. The system of claim 30, wherein the colorant is a dye.

33. The system of claim 32, wherein the dye comprises a chromophore that is at least one of: azo, carbonyl, methane, nitro, hydrazo, anthraquinone, methine, styryl, methane, cyanine, thiazine, and quinone.

34. The system of claim 32, wherein the dye is at least one of an indole, pyrazolone, pyridone, phenol, anthraquinone benzodifuranone indigoid, polymethine, styryl, carbonium, triphenylmethane, diphenylmethane, thiazine, triphendioxazine, phthalocyanine quinophthalone, nitrodiphenylamine, naphthol, stilbene, formazan, hydrazone, azine, xanthene, triarylmethane, azine, acridine, oxazine, pyrazole, pyrazoline, pyrazalone, coumarin, naphthalimide, carotenoid, flavonol, flavone, chroman, aniline black, quinacridone, formazan, triphendioxazine, thiazine, ketone amine, caramel poly(hydroxyethyl methacrylate) copolymers, riboflavin, and derivatives and mixtures thereof.

35. The system of claim 32, wherein the dye is one of a reactive dye, direct dye, mordant dye, pigment dye, anionic dye, ingrain dye, vat dye, sulfur dye, disperse dye, basic dye, cationic dye, solvent dye, and acid dye.

36. The system of claim 32, wherein the colorant-containing solution comprises a dye intermediate and wherein the dye intermediate is precipitated by the rare earth-containing composition.

37. The system of claim 32, wherein the colorant-containing solution comprises a dye carrier and wherein the dye carrier is precipitated by the rare earth-containing composition.

38. The system of claim 30, wherein the colorant is a pigment.

39. The system of claim 38, wherein the pigment is at least one of a cadmium, carbon, chromium, cobalt, copper, pyrogallol, pyrophyllite, silver, iron oxide, clay earth, lead, mercury, titanium, ultramarine, aluminum, bismuth, bronze, calcium carbonate, chromium-cobalt-aluminum oxide, cyanide iron, manganese violet, mica, zinc, spinel, rutile, zirconium, tin, cadmium, lead chromate, luminescent, metal effect, nacreous, transparent, ferric ammonium citrate, ferrous gluconate, dihydroxyacetone, guaiazulene, alizarin, alizarin crimson, gamboge, cochineal red, betacyanins, betataxanthins, anthocyanin, logwood extract, pearl essence, paprika, paprika oleoresins, saffron, turmeric, turmeric oleoresin, rose madder, indigo, Indian yellow, tagetes meal and extract, Tyrian purple, dried algae meal, henna, quinacridone, magenta, phthalo green, phthalo blue, copper phthalocyanine, indanthone, triarylcarbonium sulfonate, triarylcarbonium PTMA salt, triaryl carbonium Ba salt, triarylcarbonium chloride, polychloro copper phthalocyanine, polybromochlor copper phthalocyanine, azo, pyrazolone, monoazo benzimid-azolone, perinone, naphthol AS, beta-naphthol red, naphthol AS, disazo pyrazolone, BONA, beta naphthol, triarylcarbonium PTMA salt, disazo condensation, anthraquinone, perylene, diketopyrrolopyrrole, dioxazine, diarylide, isoindolinone, quinophthalone, isoindoline, monoazo benzimidazolone, monoazo pyrazolone, disazo, benzimidazolones, diarylide yellow dintraniline orange, pyrazolone orange, para red, lithol, azo condensation, lake, diaryl pyrrolopyrrole, thioindigo, aminoanthraquinone, dioxazine, isoindolinone, isoindoline, and quinphthalone pigments, and mixtures thereof.

40. A method, comprising:
providing a contaminated rare earth-containing composition comprising a rare earth and a colorant sorbed on the rare earth; and
sterilizing the contaminated rare earth-containing composition to remove the colorant.

41. The method of claim 40, wherein the sterilizing comprises exposing the contaminated rare earth-containing composition to an elevated temperature.

42. The method of claim 40, wherein the sterilizing comprises exposing the contaminated rare earth-containing composition to an elevated pressure.

43. The method of claim 40, wherein the sterilizing comprises exposing the contaminated rare earth-containing composition to an elevated level of radiation, the radiation being ultraviolet, microwave, and/or ionizing radiation.

44. The method of claim 40, wherein the sterilizing comprises exposing the contaminated rare earth-containing composition to a chemical oxidant.

45. The method of claim 40, wherein the sterilizing comprises exposing the contaminated rare earth-containing composition to a chemical reductant.

46. The method of claim 40, wherein the sterilizing comprises exposing the contaminated rare earth-containing composition to a chemical species that is at least one of a reductant and oxidant.

47. The method of claim 40, wherein
sensing, by at least one of colorimetry and photometry, an optical property of the treated aqueous solution to determine when to replace the rare earth-containing composition.

48. The process of claim 47, wherein the colorant is a dye.

49. The process of claim 47, wherein the dye comprises a chromophore that is at least one of: azo, carbonyl, methane, nitro, hydrazo, anthraquinone, methine, styryl, methane, cyanine, thiazine, and quinone.

50. The process of claim 49, wherein the dye is at least one of an indole, pyrazolone, pyridone, phenol, anthraquinone benzodifuranone indigoid, polymethine, styryl, carbonium, triphenylmethane, diphenylmethane, thiazine, triphendioxazine, phthalocyanine quinophthalone, nitrodiphenylamine, naphthol, stilbene, formazan s, hydrazone, azine, xanthene, triarylmethane, azine, acridine, oxazine, pyrazole, pyrazoline, pyrazalone, coumarin, naphthalimide, carotenoid, flavonol, flavone, chroman, aniline black, quinacridone, formazan, triphendioxazine, thiazine, ketone amine, caramel poly(hydroxyethyl methacrylate) copolymers, riboflavin, and derivatives and mixtures thereof.

51. The process of claim 49, wherein the dye is one of a reactive dye, direct dye, mordant dye, pigment dye, anionic dye, ingrain dye, vat dye, sulfur dye, disperse dye, basic dye, cationic dye, solvent dye, and acid dye.

52. The process of claim 49, wherein the contaminated rare earth-containing composition comprises a dye intermediate and wherein the dye intermediate is precipitated by the rare earth composition.

53. The process of claim 49, wherein the contaminated rare earth-containing composition comprises a dye carrier and wherein the dye carrier is precipitated by the rare earth composition.

54. The process of claim 47, wherein the colorant is a pigment.

55. The process of claim 54, wherein the pigment is at least one of a cadmium, carbon, chromium, cobalt, copper, pyrogallol, pyrophyllite, silver, iron oxide, clay earth, lead, mercury, titanium, ultramarine, aluminum, bismuth, bronze, calcium carbonate, chromium-cobalt-aluminum oxide, cyanide iron, manganese violet, mica, zinc, spinel, rutile, zirconium, tin, cadmium, lead chromate, luminescent, metal effect, nacreous, transparent, ferric ammonium citrate, ferrous gluconate, dihydroxyacetone, guaiazulene, alizarin, alizarin crimson, gamboge, cochineal red, betacyanins, betataxanthins, anthocyanin, logwood extract, pearl essence, paprika, paprika oleoresins, saffron, turmeric, turmeric oleoresin, rose madder, indigo, Indian yellow, tagetes meal and extract, Tyrian purple, dried algae meal, henna, quinacridone, magenta, phthalo green, phthalo blue, copper phthalocyanine, indanthone, triarylcarbonium sulfonate, triarylcarbonium PTMA salt, triaryl carbonium Ba salt, triarylcarbonium chloride, polychloro copper phthalocyanine, polybromochlor copper phthalocyanine, azo, pyrazolone, monoazo benzimid-azolone, perinone, naphthol AS, beta-naphthol red, naphthol AS, disazo pyrazolone, BONA, beta naphthol, triarylcarbonium PTMA salt, disazo condensation, anthraquinone, perylene, diketopyrrolopyrrole, dioxazine, diarylide, isoindolinone, quinophthalone, isoindoline, monoazo benzimidazolone, monoazo pyrazolone, disazo, benzimidazolones, diarylide yellow dintraniline orange, pyrazolone orange, para red, lithol, azo condensation, lake, diaryl pyrrolopyrrole, thioindigo, aminoanthraquinone, dioxazine, isoindolinone, isoindoline, and quinphthalone pigments, and mixtures thereof.
